(19) United States
(12) Patent Application Publication
(10) Pub. No.: US 2019/0352415 A1
(43) Pub. Date: Nov. 21, 2019

(54) USE OF THE CD71 RECEPTOR IN THE PROGNOSIS AND TREATMENT OF ENDOMETRIOSIS

(71) Applicants: ENDODIAG, Evry (FR); INATHERYS, Evry (FR)

(72) Inventors: Pierre LAUNAY, Rueil Malmaison (FR); Coralie BELANGER, Paris (FR); Cécile REAL, Paris (FR)

(73) Assignees: ENDODIAG, Evry (FR); INATHERYS, Evry (FR)

(21) Appl. No.: 16/478,979

(22) PCT Filed: Jan. 19, 2018
(86) PCT No.: PCT/FR2018/050140

§ 371 Date: Jul. 18, 2019

(30) Foreign Application Priority Data
Jan. 20, 2017 (FR) 17/50468

(51) Int. Cl. C07K 16/28; A61P 15/08; G01N 33/68

(52) U.S. Cl. CPC C07K 16/2881; A61K 2039/505; G01N 33/6893; A61P 15/08

(57) ABSTRACT

The CD71 receptor is used as a target in the prognosis and/or treatment of endometriosis, and to a test for the prognosis or therapeutic monitoring of endometriosis, targeting this receptor.

Endometriosis is caused by a tissue similar to the endometrial tissue that grows out of the uterus and causes lesions, adhesions, and cysts in the colonized organs. Characterized by intense pain, it affects 10% of women in childbearing age, namely 180 million women worldwide (Abbott, 2013, ASRM, 2012, Bulletti et al., 2010, Nucci and Oliva, 2009).

Endometriosis may be ovarian, peritoneal, intestinal, rectal, and sometimes even, in some rare forms, umbilical, pulmonary or cutaneous.

For the concerned women, the main symptoms are menstrual cramps, disabling chronic pain, painful urination and very irregular and excessive menstruations. The disease has a strong impact on the social and professional life of these women (ESHRE, 2007). Infertility is also a major consequence of endometriosis: it is estimated that up to 40% of women having endometriosis are infertile; conversely, nearly 40% of cases of infertility are due to endometriosis.

Despite a very high incidence and prevalence, endometriosis is diagnosed with an average delay of nine years, during which the disease has had time to cause significant damage to the different affected organs.

The annual societal cost of endometriosis has been estimated only for the United States at 49.6 billion dollars (Simoens et al, 2012). This cost is comparable to that of other chronic diseases such as type II diabetes or Crohn's disease.

In France, the annual cost per patient was extrapolated to 10,000 euros in 2012 from the estimated cost in Italy by Simoens (Official Journal of the Senate, April 2013).

Current Treatments of Endometriosis

According to the American Society for Reproductive Medicine (ASRM), endometriosis must be considered as a chronic disease requiring lifelong medical monitoring of the patients having the disease in order to prioritize medical treatments and avoid repeated surgical procedures. The ASRM estimates that 40 to 80% of women suffering from endometriosis suffer from recurrent chronic pain within two years of surgery and that long-term monitoring of the pain associated with endometriosis is usually required. (ASRM, 2012).

Although no specific drug currently exists for endometriosis, several approaches are used to treat the symptoms associated with this pathology. Treatment options comprise surgery, hormonal treatments, or a combination thereof.

Conservative surgery is a frequently used option for women who want to become pregnant. Indeed, endometriosis is one of the main factors of infertility. Due to the high rate of post-surgical recurrence of endometriosis, management most often combines surgical and medical aspects to improve results (RCOG, 2006).

A medical treatment is generally prescribed in cases where the pain associated with endometriosis significantly affects the quality of life of the patient.

The most commonly used endometriosis treatments are the following ones:
Progestatives and Combined Oral Contraceptives (COCs)
LH-RH agonists. This treatment cannot be prescribed beyond a period of one year.
Nonsteroidal anti-inflammatory drugs
Danazol
Operative coelioscopy (most used treatment)
Hysterectomy with ovariectomy and removal of all lesions in place to prevent any risk of recurrence (often the “last resort” operation).

To date, there is no cure for endometriosis.

Because of their favorable safety, tolerability and cost profile, COCs are consistently considered as the first-line treatment option, both as an alternative to surgery and as a postoperative adjuvant. These drugs block ovulation and inhibit the growth of endometriomas. COCs are generally well tolerated and have few side effects. Progestatives take on many forms, including intrauterine devices (IUDs), injections, and the pill. Non-steroidal anti-inflammatory drugs (NSAIDs) are generally used in conjunction with first-line treatments to relieve pain. Danazol is a derivative of ethisterone, and was the first drug approved in the United States to treat endometriosis. Although it is effective in reducing the size of endometriotic implants, its excessive side effects (virilization) make it almost no longer in use.

Today, GnRH agonists are considered as the first-line medical treatment for the pain associated with moderate-to-severe endometriosis. However, their side effects and in particular the significant loss of bone density they cause have led to the limitation of this treatment over time to a maximum of 12 months.

The problem with hormone treatments is that, by definition, their use prevents the conception. Indeed, many patients discover that they suffer from endometriosis during a checkup for infertility. However, most of the treatments currently offered are hormonotherapy targeting the oestrogen production pathway, and therefore often contraceptives.

Because of the intrinsic limitations of the available treatments, there is therefore a very considerable need for innovative drugs with new mechanisms of action independent of hormonotherapy to treat patients suffering from endometriosis.

Endometriosis usually decreases and disappears after menopause, but should still be monitored especially when substitute hormone therapies are put in place at menopause.

The CD71 Receptor

CD71 is a transferrin receptor present at the surface of all human cells. Transferrin is a protein enabling the transport of an element essential to life, iron, within the body. Transferrin binds to its receptor CD71 and thus enables the penetration of iron into the cell.

The more a cell multiplies, for example the cancer cell or the endometriotic cell, the more its need for iron increases. Several studies show that tumor cells, with high proliferative capacity, strongly express the CD71 receptor, thus allowing iron uptake adapted to the needs of the cell. The CD71 key regulator of cell activity, and more particularly of cell proliferation, therefore appears to be a relevant target for blocking tumor proliferations and eradicating cancer cells.

The CD71 receptor a new target in the treatment of endometriosis CD71 regulates cell activation and proliferation by enabling the entry of iron into the cell. Preclinical studies conducted by Inatherys have shown that tumor cells with high proliferative capacity strongly express the CD71 receptor and that the blocking of its biological activity by the anti-CD71 antibody by inducing an iron deficiency of the cell, inhibits the proliferation of tumor cells and causes cell death by apoptosis. Inatherys has also demonstrated the specificity of the targeting of the anti-CD71 antibody which is preferentially bound on cells with a high density of transferrin receptors. This is in particular the object of the application WO 2017/013230A1.

It has recently been shown in several international publications that iron metabolism is involved in the pathogenesis of endometriosis, and that iron is present at high concentrations in the peritoneal cavity of patients (Defrère et al., 2008—Molecular Human Reproduction Vol. 14, No. 7 pp. 377-385, 2008; Lousse et al., 2009—Fertility and Sterility Vol 91, No. 5, May 2009; Sanchez et al., 2014—Human Reproduction, Vol. 29, No. 3 pp. 577-583, 2014).

Thus, demonstrating that similarly to cancer cells, endometriotic cells overexpress the CD71 receptor at their surface to address their increased need for iron and then demonstrate the antiproliferative activity of the anti-CD71 antibody on these cells is a new therapeutic strategy with high potential in endometriosis.

Use of an Anti-CD71 Antibody: An Innovative Mechanism of Action to Target CD71 and a Promising Treatment of Endometriosis

Such an antibody essentially targets cells with a high proliferative capacity: it competes with transferrin for its binding to CD71. Under physiological conditions, the quiescent cells have a limited number of CD71 molecules at their surface favoring transferrin binding. In contrast, rapidly growing cancer cells or endometriotic cells have an increased need for iron and thus a high density of CD71 receptors at their surface. In this case, the anti-CD71 antibody bivalently binds to CD71 and prevents the binding of transferrin to its receptor. The anti-CD71 antibody:
reduces the number of CD71s at the cell surface. It induces internalization of the receptor towards the lysosomal compartments where it is degraded. This very rapid decrease involves up to 90% of the receptors present at the cell surface,
deprives the tumor cells of iron. The tumor or endometriotic cells targeted by the anti-CD71 antibody therefore have a very significant reduction in iron intake. This iron deprivation appears to be the major mechanism involved in the death of cancer cells.
acts autonomously. In contrast to most commercially available monoclonal antibodies, the anti-CD71 antibody has an intrinsic and independent effect on antibody-dependent cellular cytotoxicity (ADCC) or complement-dependent cytotoxicity (CDC) and thus on the immune status of the patient.

Thus, an object of the invention is an anti-CD71 antibody for its use in the treatment of endometriosis, as well as a therapeutic treatment method comprising the administration of an anti-CD71 antibody to a subject suffering from endometriosis and eligible for said treatment.

The term antibody means in particular a monoclonal antibody or fragment, as defined in the document WO 2017/013230A1.

The CD71 Receptor a New Target in the Diagnosis of Endometriosis

In parallel with this therapeutic innovation, the development of a diagnosis test targeting the CD71 receptor makes it possible to identify the patients who can respond to this new treatment. The evaluation of CD71 receptor density at the level of the endometriotic lesions constitutes a companion diagnostic test allowing validating, on a case-by-case basis, the use of an anti-CD71 antibody as a treatment against endometriosis.

This test may take on the form of an Immunohistochemistry (IHC) kit comprising the anti-CD71 antibody as an antibody specific for detecting the expression of the transferrin receptor. Immunohistochemistry being a technique routinely used in medical analysis laboratories or hospital laboratories, allowing carrying out semi-quantitative analyses directly on tissues in order to provide information on the presence and location of the targeted receptors, it is a technique of choice to develop a robust, reliable, inexpensive and quick to implement in vitro diagnosis kit.

In the manner of Herceptest, in breast cancer which stratifies patients according to the overexpression of Her2neu to determine which ones will benefit from a treatment with Herceptin, a diagnosis test assessing the overexpression of CD71 provides a real paradigm shift in treating patients with endometriosis. Indeed, until then the choice of a treatment was not targeted. Defining the treatment rationally by predicting its effectiveness is a significant improvement in the management of patients.

In addition, besides the selection of the patients eligible for this treatment, this companion test will allow in the short term:
Improving the rate of response to anti-CD71 antibodies in endometriosis during clinical development by targeting responder patients
Improving the benefit/risk ratio
Reducing the size of the necessary cohorts thanks to the stratification of the patients which makes it possible to target only potentially responder patients without losing statistical power
Decreasing the duration of the development phase of the anti-CD71 antibody through smaller cohorts in phase II and III as well as associated costs

Before discussing the different objects of the invention, certain terms appearing in the present text are defined.

The diagnosis consists in detecting in a biological sample of a tested subject, whether or not (s)he has an endometriosis.

The prognosis consists in determining the risk for a subject whose diagnosis of endometriosis has already been made to have his disease evolve quickly or not. We talk about proliferative, or barely proliferative, endometriosis depending on the level of expression of the CD71 receptor.

The prediction consists in selecting among subjects having endometriosis those whose biological characteristics make it possible to predict whether a targeted treatment will be effective or not.

The therapeutic monitoring consists in determining whether or not a subject having endometriosis and treated for this endometriosis responds to treatment.

The term biological sample means any sample likely to contain the CD71 receptor. It may originate from a sampling of any biological fluid, such as whole blood, serum, plasma, urine, cerebrospinal fluid, organic secretions, saliva, effusions, stool, bone marrow, and cells purified from these liquid samples, or a tissue specimen or a tissue, or isolated cells. Preferably, it consists of an endometriotic lesion that has been surgically removed, usually by laparoscopy.

Thus the object of the present invention is:
The use of the CD71 receptor as a target in the detection and/or treatment of endometriosis.
A diagnosis test targeting the CD71 receptor in a biological sample for the detection and/or treatment monitoring of endometriosis.
A diagnosis test allowing assessing the overexpression of the CD71 receptor.
A diagnosis or therapeutic monitoring kit comprising a specific antibody of the CD71 receptor.

In a particular implementation, the diagnosis kit further comprising antibodies targeting the KI67 marker and/or the Bcl2 protein.

In another particular implementation of the invention, the diagnosis or therapeutic monitoring kit is an immunohistochemistry kit.
The use of the kit according to the invention for detecting and/or following-up the treatment of endometriosis.
The anti-CD71 antibody, or a molecule targeting the CD71 receptor, for its use in the detection and/or treatment of endometriosis.
A method for detecting the degree/severity of endometriosis by measuring the concentration/density/expression of the CD71 receptor; more specifically, this method is carried out in vitro by comparative measurement of the concentration/density/expression of CD71 between a healthy subject and a subject having endometriosis by immunohistochemistry implementing an anti-CD71 antibody.

Advantageously, the invention concerns the use of an anti-CD71 antibody for the prognosis of endometriosis, as well as a method for prognosis of endometriosis comprising contacting a biological sample of a subject having endometriosis with an anti-CD71 antibody. In particular, this use or method is intended for the prognosis of a development of a hyperproliferative endometriosis overexpressing the CD71 receptor.

It also concerns the use of an anti-CD71 antibody for predicting success in a treatment of endometriosis, as well as a method for predicting success in the treatment of endometriosis. In particular, this use or method is intended for the prediction of success in a treatment of endometriosis overexpressing the CD71 receptor.

The invention further concerns a prognosis, predictive or therapeutic monitoring test of endometriosis in a subject having endometriosis, comprising contacting an anti-CD71 antibody with a biological sample of said subject.

In the context of this test, the invention relates to a prognosis, predictive or therapeutic monitoring kit comprising an anti-CD71 antibody. Advantageously, a kit further comprises antibodies targeting the KI97 marker and/or the Bcl2 protein. A preferred kit of the invention is an immunohistochemistry kit.

### EXAMPLE 1: PROOF OF CONCEPT OF THE USE OF THE ANTI-CD71 ANTIBODY AS A POSSIBLE TREATMENT OF ENDOMETRIOSIS

1) Validation of the Presence of Transferrin Receptors on Retrospective Endometriotic Lesions Embedded in Paraffin

This step was conducted on endometriosis samples embedded in paraffin (n=44) thanks to the immunohistochemistry (IHC) technique, a technique that Endodiag routinely uses. Each series of sample blocks was representative of a stage of the pathology.

In parallel with the validation of the presence of CD71 at the level of the endometriosis lesions, IHCs targeting the proliferation marker KI67 and the oncogene Bcl2 were carried out. Indeed, the anti-CD71 antibody preferably targets cells with a high rate of proliferation and induces their apoptosis by iron deprivation. The cells targeted by the anti-CD71 antibody, in addition to expressing CD71, are positive for the proliferation factor KI67 and the anti-apoptotic marker Bcl2.

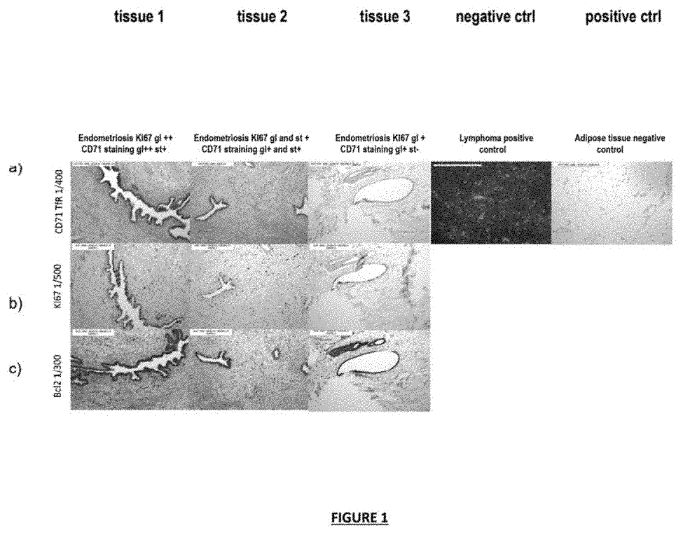

FIG. 1 shows:
a) anti CD71/transferrin receptor IHC,
b) anti KI67 (proliferation marker) IHC,
c) anti Bcl2 (anti-apoptotic marker) IHC.

The IHCs were carried out on 44 biopsies of endometriotic lesions. The results demonstrated that the intensity of CD71 staining is correlated with that of KI67. Thus, the higher the KI67 staining (gland and/or stroma), the higher the CD71 staining will be (tissue No. 1). Conversely, for lesions having low KI67 staining, CD71 staining is also barely intense (tissue No 3).

There is also a correlation between the KI67 and CD71 stainings and that of Bcl2. The higher the KI67 or CD71 staining, the higher the bcl2 staining will be. This confirms the proliferative status of endometriotic cells (high proliferation and inhibited apoptosis).

The most proliferative endometriotic lesions (associated with high KI67) also overexpress the CD71 receptor. There is a correlation between the expression of these two markers. This first result makes it possible to assume that, like cancer cells, these proliferative lesions would be sensitive to the targeted action of the anti-CD71 antibody.

These tests made it possible to validate the interest of testing the anti-CD71 antibody in the treatment of endometriosis.

### EXAMPLE 2: DEMONSTRATION OF THE CORRELATION BETWEEN THE CD71 DENSITY AT THE SURFACE OF ENDOMETRIOTIC CELLS AND THE THERAPEUTIC EFFECTIVENESS OF THE ANTI-CD71 ANTIBODY

a) The first part begins during in vitro tests carried out on primary cultures of cells originating from endometriotic lesions. It consists in testing the effect of the anti-CD71 antibody on these cultures (test of proliferation, apoptosis and invasiveness, dose-dependent) and checking on the anti-proliferative effect of the anti-CD71 antibody. Immunocytochemistry with the anti-CD71 antibody is carried out in parallel to quantify the CD71 receptors at the cell surface. Flow cytometry experiments are also carried out to quantify the CD71 receptors at the cell surface. These data are analyzed in parallel with the effect observed on the cells in vitro and a correlation between CD71 density and the anti-CD71 antibody efficiency is looked for.

b) Secondly, this correlation is demonstrated during in vivo tests on a mouse model of endometriotic cell xenograft. The different tests carried out on this model make it possible to highlight the effectiveness of the anti-CD71 antibody on the endometriotic cells. These results are then correlated with the CD71 receptors density at the level of the endometriotic lesions in the xenografted mouse and are analyzed by IHC at the end of the experiment.

### EXAMPLE 3: IN VIVO TESTS

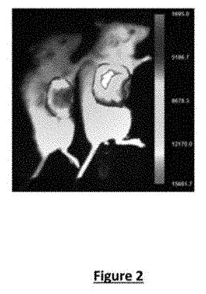

Once the in vivo technique has been validated, endometriotic cells originating from 30 dissociated fresh lesions are injected and a series of tests is carried out using this model with the use of multiple doses of the anti-CD71 antibody, ranging from 1, 5, 10, 20 and 40 mg/kg in one single peritoneal injection, as we previously validated for hematological malignancies. Then several modes of administration are tested, intraperitoneal (I.P) and intravenous (I.V) as well as several doses of this treatment: once a week to 3 times a week. This study allows us to validate the use of the anti-CD71 antibody in vivo for the treatment of endometriosis and to determine a theoretical dose to be used in the first toxicology tests in monkeys. In order to be able to accurately quantify the inhibition of cell proliferation following the treatment with the anti-CD71 antibody, a Xenolight RediJect 2-DG-750 probe that allows visualizing the glucose metabolism within the tumor (synonym of proliferation) is injected to monitor cell proliferation in vivo. Fluorescence is measured using Kodak's imagine FX pro in vivo detector and images are analyzed by Carestream MI software (FIG. 2). Experimental studies on animals are subject to referral to the ethics committee and are validated by Paris V university regulatory authorities.

FIG. 2 shows an example of following-up the subcutaneous development of xenografts in NSG mouse by microscopic imaging. Differences in cell proliferation are accounted by subtracting fluorescences between treated and control animals

### EXAMPLE 4: DESCRIPTION OF THE KIT

The kit is an immunohistochemistry (IHC) kit based on the use of the anti-CD71 antibody, thus guaranteeing sensitivity and selectivity. The anti-CD71 antibody intended for the diagnosis use differs from the anti-CD71 antibody intended for therapeutic use only by the production method that will not be done under the strict framework of GMP (Good Manufacturing Practices). The clones producing the anti-CD71 will nevertheless be identical.

The anti-CD71 IHC kit makes it possible to carry out between 30 and 50 tests, which is generally proposed for commercially available IHC kits.

It comprises all the reagents necessary for carrying out the test as well as the positive and negative control slides and a complete protocol with the elements necessary for the analysis of the result.

### EXAMPLE 5: OPTIMIZATION OF THE TEST

It consists in defining the optimal conditions and the limiting conditions of use of the kit. This phase requires the use of twenty fresh specimens of endometriosis lesions.

This step covers respectively:
the pre-analytical steps that represent all the steps of the process preceding the analysis of the tissues namely, tissue sampling, transport, fixation, inclusion, cutting thickness, slide type, etc. until the completion of the section of the paraffin embedded block.
The actual steps of the analysis namely all steps of the IHC protocol, staining and mounting of the slides
The post-analytical steps which comprise the interpretation and the renderings of results.

During the pre-analytical phases allow defining the following criteria and variables:
Method for sampling tissue
Fixation (time, method)
Decalcification (time, method)

These steps are critical because the lesions sampling conditions can vary significantly from one surgeon to another and lead to sample degradation. For example, the use of pliers and scissors makes the samples unanalyzable in 40% of cases.

These phases have already been optimized, thanks to the development of an endometriosis biopsy gun and a sampling kit containing all the elements necessary for the rapid fixation of biopsies (see WO2013/034573).

The analytical phases make it possible to define the following criteria and variables:
Thickness of cut
Slide type
dewaxing method
antigen unmasking method
visualization system
reagents
controls
antibodies
method for carrying out the test (automatic, manual)
staining method
type of lamella mounting

In more detail, it is a question of validating all the conditions of completion of the test and the reagents which are used during the latter in order to obtain a satisfactory staining, a properly colored and mounted tissue and therefore optimally analyzable. For each parameter to be adjusted, the various methods commonly used are tested and the most appropriate one is selected.

Thus, the following data are evaluated:
the impact of the thickness of the cut on the results,
the impact of the slide type (simple slide, superfrost+® slide, FLEX® slide),
the impact of the dewaxing method (time, temperature, material, coupled or not with the antigen unmasking),
the impact of the visualization system; kit based on biotin/streptavidin or on a polymer, type of chromogen used (DAB, AEC), type of enzyme (HRP or AP), etc.,
the impact of the reagents (washing buffer with or without detergent, blocking of endogenous peroxidase, etc.),
appropriate controls (positive and negative internal and/or external controls),
The concentration and optimal antibody staining time required to obtain high quality detection and staining and the most appropriate diluent
the stability of the antibody from 0 to 6 months
the stability of the other selected reagents
the used method, manual or using an IHC automaton
the time and method of counterstaining (hematoxylin from Mayer, Harris)
the type of coverslip mounting (permanent, aqueous, according to the selected chromogen)

1.-10. (canceled)

11. A method of treating endometriosis, comprising administering to a subject having endometriosis, an anti-CD71 antibody or a molecule targeting the CD71 receptor.

12. A method for the prognosis of endometriosis, or for predicting or monitoring success of a treatment of endometriosis, in a subject having endometriosis, comprising contacting an anti-CD71 antibody with a biological sample of said subject.

13. The method according to claim 12, for the prognosis of a development of an hyperproliferative endometriosis overexpressing the CD71 receptor.

14. The method according to claim 12, for predicting success of a treatment of endometriosis overexpressing the CD71 receptor.

15. A prognostic, predictive or therapeutic monitoring kit, comprising an anti-CD71 antibody.

16. The kit according to claim 15, further comprising antibodies targeting the KI67 marker and/or the Bcl2 protein.

17. An immunohistochemistry kit according to claim 15.

18. A method for the prognosis of endometriosis, according to claim 12, which is for in vitro detection of the degree/severity of endometriosis by comparative measurement of the concentration/density/expression of CD71 between a healthy subject and a subject having endometriosis by immunohistochemistry implementing an anti-CD71 antibody.
